(19) United States
(12) Patent Application Publication
(10) Pub. No.: US 2009/0131796 A1
(43) Pub. Date: May 21, 2009

(54) APPARATUS AND METHOD FOR MODIFYING ULTRASONIC TISSUE HARMONIC AMPLITUDE

(75) Inventor: Che-chou Shen, Taipei City (TW)

Correspondence Address: KIRTON AND MCCONKIE, 60 EAST SOUTH TEMPLE,, SUITE 1800, SALT LAKE CITY, UT, 84111, US

(73) Assignee: National Taiwan University of Science and Technology, Taipei City (TW)

(21) Appl. No.: 12/145,075

(22) Filed: Jun. 24, 2008

(30) Foreign Application Priority Data
Nov. 20, 2007 (TW) 096143860

(51) Int. Cl. A61B 8/14

(52) U.S. Cl. 600/445

(57) ABSTRACT

An ultrasonic diagnostic apparatus and imaging method for harmonic enhancement or suppression are disclosed. A fundamental transmit signal and a 3f₀ transmit signal are combined to emit as an ultrasonic pulse signal. For harmonic enhancement, when the phase of 3f₀ transmit signal is adjusted relative to that of the fundamental transmit signal, the harmonic signal is effectively enhanced due to the frequency-sum and frequency-difference components being in-phase for constructive combination. For harmonic suppression, the 3f₀ phase can be further adjusted for about 180 degrees from that of maximal enhancement. The harmonic signal is effectively suppressed due to the two components being out-phase for destructive cancellation. With the apparatus, both harmonic enhancement and harmonic suppression can be achieved for better image quality in ultrasonic harmonic imaging.

This patent application claims the national priority from Taiwan patent application, Ser. No. 096143860, filed on Nov. 20, 2007. This invention is disclosed by an published article, Che-Chou Shen, et al, “THIRD HARMONIC TRANSMIT PHASING FOR TISSUE HARMONIC GENERATION,” pp. 1370-1381, IEEE transactions on ultrasonics, ferroelectrics, and frequency control, vol. 54, NO. 7, July 2007.

### TECHNICAL FIELD OF THE INVENTION

The present invention relates to an apparatus and method for improving ultrasonic image quality for medical purposes, and more particularly, to an apparatus and method that modify the amplitude of tissue harmonic signals to improve the image quality.

### BACKGROUND OF THE INVENTION

Various imaging techniques are applied in medicine for clinical diagnosis, for example, X-ray imaging, ultrasonic imaging, computed tomography scan (CT scan) and magnetic resonance imaging (MRI) etc. Among them, ultrasonic imaging has its superiority. Generally, an ultrasonic diagnostic apparatus is comparatively small in size, inexpensive and has ability of providing instant images. In addition, it is safe for patients to be examined since the ultrasonic examination is noninvasive and he or she is free from exposing in radiation (for example, X-ray). Moreover, by combining a series of static ultrasonic images, dynamic ones (for example, bloodstream, heart beating) are therefore observable.

ultrasonic beams propagated in a medium (for example, tissue) generate echo signals. The echo signals are converted into electronic signals and then processed to display on a screen as an ultrasonic image. During acoustic propagation in a tissue, since reflection/scatter, refraction or frequency response on interfaces causes a portion of beam energy to be absorbed or scattered, the amplitude of echo signals is reduced. Since the amplitude differs for different density distribution of tissue, the received echo signals are converted into corresponding amplitude of electronic signals. The electronic signals mostly include noises. To eliminate the noises, the signals are analyzed with artificial intelligence (AI) dynamically. Another way is to truncate a range of frequency with a filter, keeping the necessary frequency range of signals. The ultrasonic image is plotted with the time sequences that the electronic signals are received, representing depth in the tissue, and the amplitude being brightness on the screen.

Generally speaking, ultrasonic frequency used in medical applications reaches several megahertz (MHz). The ultrasonic wave is a mechanical wave that has positive correlations between attenuation and frequency. That is, high frequency leads to quick attenuation and short propagation distance. Conversely, low frequency leads to slow attenuation and long propagation distance. However, since an ultrasonic wave with a high frequency has more wavenumbers, more computable data are provided, resulting in better image resolution. Generally, low frequency is employed to probe deep positions for reducing signal decay, while high frequency is utilized at shallow positions for high resolution consideration.

Referring to FIG. 1, a frequency f₀ of 2 MHz is used for scanning a biological body. When processing echo signals of the 2 MHz frequency for image formation, a so-called fundamental imaging can be obtained. On the other hand, processing of harmonic signals of higher frequencies, 4, 6, or 8 MHz for imaging, is referred to as harmonic imaging. These harmonic signals are produced either due to finite amplitude distortion when sound wave propagates through a human body or due to the harmonic oscillation of microbubble contrast agents. Since the harmonic signal is generated from the nonlinearity of the imaged objects, harmonic imaging is also called nonlinear imaging.

Conventionally, the ultrasonic image is produced by linearly backscattered fundamental signals. Since the tissue harmonic amplitude is lower than the amplitude of the fundamental signal in the beginning of acoustic propagation, the tissue harmonic signal suffers less from phase aberration and reverberation from shallow structures that deteriorate image quality. Therefore, the tissue harmonic imaging is superior to the conventional fundamental imaging with higher contrast resolution. As a result, the tissue harmonic imaging is widely used in clinical applications.

To extract harmonic signals, a low-pass or high-pass filer is utilized to select a range of signal frequency in order to obtain a desired harmonic image. Generally, the second harmonic signal is the dominant component in harmonic imaging due to its highest amplitude among the received harmonics. However, the amplitude of the second harmonic signal is still much weaker than the fundamental amplitude, and therefore, both penetration and sensitivity of tissue harmonic imaging are limited. Consequently, it is necessary to enhance the harmonic amplitude to improve image quality.

The other kind of harmonic imaging, the contrast harmonic imaging, is performed with injecting microbubble contrast agents into blood vessel to achieve image enhancement. The contrast agent is composed of thousands of microbubbles. These microbubbles resonate when being excited by sound waves and produce strong harmonic signal. However, this technology is sometimes subject to limitation because significant tissue harmonic signals are also present in the region of surrounding tissues. Under this circumstance, it is hard to identify the position of blood vessels. Therefore, some other techniques lay stress on suppressing the tissue harmonic signals to increase the contrast-to-tissue ratio (CTR) between the surrounding tissues and the blood vessel. Consequently, both harmonic enhancement and harmonic suppression are helpful for ultrasonic imaging.

For harmonic enhancement, a conventional method called coded excitation is used to extend the time period to transmit more acoustic energy into tissue to boost harmonic generation. The amplitude of the second harmonic signal is thus enhanced. To avoid damaging tissues, the peak amplitude of the ultrasonic signal is emitted at acoustic pressure within current safety regulations. Generally, the axial resolution in coded excitation is degraded due to elongated transmission waveform; hence, a pulse compression filter would be required to restore the resolution. However, pulse compression will generate sidelobes, which reduce image quality.

Method of multiple transmit focusing also increases the harmonic amplitude with a plurality of focal depths. In addition to an original focal depth, it can be set to focalize at other positions to increase the harmonic amplitude therein. However, the disadvantage of the multiple transmit focusing method is that it increases sidelobes, leading to a lower contrast resolution. This is because the beam of main focal point will interfere with the beam of second focal point. Sidelobes within the main focal beam is thus increased.

For harmonic suppression, a conventional method called source prebiasing is to combine the fundamental component with a 2f₀ signal to form an emitting waveform. The 2f₀ signal, after nonlinear propagating, can eliminate the second harmonic signal generated by the traditional method (only the fundamental signal emitted). To acquire the 2f₀ signal, the second harmonic signal is firstly extracted and inverted. The inverted signal is linearly backpropagated to the surface of the transducer to obtain the desired 2f₀ signal. Finally, the 2f₀ signal is joined to the fundamental component to provide an emitting signal. However, in the near field, harmonic signals obtained by source prebiasing are higher than signals obtained by the traditional method (only the fundamental signal emitted) and it will affect the contrast agents detection.

Therefore, there is a need to develop a strategy for improving ultrasonic image quality in medical applications.

### SUMMARY OF THE INVENTION

In an aspect of the present invention, a method of third harmonic transmit phasing is provided to improve ultrasonic image quality. In the aforesaid method, a fundamental transmit signal and a 3f₀ transmit signal are emitted at the same time. The amplitude of second harmonic signal is adjusted by altering the relative phase difference between the 3f₀ transmit signal and the fundamental transmit signal. The second harmonic signal is used for harmonic imaging.

In another aspect of the present invention, for enhancement of the second harmonic signal, when the phase of the 3f₀ transmit signal is adjusted to a value, due to the frequency-sum and frequency-difference component be in-phase for constructive combination, the amplitude of the second harmonic signal is effectively enhanced. For example, when the phase of the 3f₀ transmit signal approaches triple the phase of the fundamental transmit signal, i.e. the phase difference between the 3f₀ transmit signal and the fundamental transmit signal is roughly double the phase of the fundamental transmit signal, the second harmonic signal is enhanced maximally.

In another aspect of the present invention, for suppression of the second harmonic signal, when the phase of the 3f₀ transmit signal reaches maximal enhancement, its phase is further adjusted to a degree, for example, about 180 degrees. Due to the frequency-sum and frequency-difference component be out-phase for destructive cancellation, the amplitude of the second harmonic signal is effectively suppressed. In another case to suppress the second harmonic signal, a relation is applied with 2θ=φ+π, where θ denotes the phase of the fundamental transmit signal and φ denotes the phase difference between the fundamental transmit signal and the 3f₀ transmit signal. The third harmonic transmit phasing method proposed in the present invention can suppress tissue harmonic signals so as to improve image contrast in the area perfused with a contrast agent when contrast harmonic imaging is applied.

An ultrasonic diagnostic apparatus is employed according to the present invention. The aforesaid apparatus is used for emitting an ultrasonic pulse signal to scan a biological sample. The ultrasonic pulse signal is reflected/scattered backwardly by the biological sample to result in an ultrasonic echo signal. The aforesaid apparatus comprises a transmission waveform controller for generating a combinative signal and generating a drive voltage according to the combinative signal, wherein the combinative signal comprised of a fundamental transmit signal and a 3f₀ transmit signal, which the focal frequency are fundamental (f₀) and 3f₀, respectively; a pulse generator for generating a pulse signal according to the drive voltage, wherein the pulse signal is converted to the ultrasonic pulse signal by piezoelectric materials; a signal receiver for receiving the ultrasonic echo signal, which comprises the reflected/scattered fundamental transmit signal and an harmonic signal; and a data processor for processing the harmonic signal of the ultrasonic echo signal to generate an image data.

An ultrasonic imaging method is employed according to the present invention. The aforesaid method is utilized to an ultrasonic pulse signal to scan a biological sample. The ultrasonic pulse signal is reflected/scattered backwardly by the biological sample to result in an ultrasonic echo signal. The aforesaid method comprises steps of generating a combinative signal, wherein the combinative signal comprised of a fundamental transmit signal and a 3f₀ transmit signal, which the focal frequency are fundamental (f₀) and 3f₀, respectively; converting the combinative signal to the ultrasonic pulse signal, wherein after emitting the ultrasonic pulse signal, the biological sample is scanned regionally; receiving the ultrasonic echo signal, which comprises the reflected/scattered fundamental transmit signal and an harmonic signal; and processing the harmonic signal of the ultrasonic echo signal to generate an image data.

### BRIEF DESCRIPTION OF THE DRAWINGS

FIG. 1 is a diagram illustrating a fundamental signal before/after finite amplitude distortion.

FIG. 2 is a functional block illustrating an ultrasonic diagnostic apparatus in the embodiment according to the present invention.

FIG. 3 is a functional block illustrating a signal converter of ultrasonic diagnostic apparatus in the embodiment according to the present invention.

FIG. 4 is a flow chart illustrating an ultrasonic imaging method in accordance with the present invention.

FIG. 5a is a diagram illustrating a second harmonic signal with the frequency-sum component and frequency-difference in the axial position.

FIG. 5b is a diagram illustrating a second harmonic signal with the frequency-sum component and frequency-difference in the lateral position.

FIG. 6 is a diagram illustrating the phase difference between a fundamental transmit signal and a 3f₀ transmit signal configuring the second harmonic signal.

FIG. 7a is a diagram illustrating enhancement and suppression of second harmonic signals in the axial position with/without emitting a 3f₀ transmit signal.

FIG. 7b is a diagram illustrating enhancement and suppression of second harmonic signals in the lateral position with/without emitting a 3f₀ transmit signal.

FIGS. 8a-1, 8a-2, and 8a-3 are diagrams illustrating that a fundamental signal with a 3f₀ transmit signal emitted is not enhanced or suppressed in the axial position.

FIGS. 8b-1, 8b-2, and 8b-3 are diagrams illustrating that a fundamental signal with a 3f₀ transmit signal emitted is not enhanced or suppressed in the lateral position.

### DETAILED DESCRIPTION OF THE INVENTION

In the present invention, an emitting signal includes fundamental and 3f₀ frequency components simultaneously. The amplitude of second tissue harmonic signal is adjusted by altering the relative phase difference between a 3f₀ transmit signal and a fundamental transmit signal. Both enhancement and suppression of second tissue harmonic signal are helpful for improving ultrasonic image quality. The mechanism of third harmonic transmit phasing method proposed in accordance with the present invention will be described in the following.

Harmonic generation is produced due to linear and nonlinear propagating of ultrasonic waves in a medium. It can be represented as Eq. (1):

$$\begin{matrix}{u}_{n} ( z + Δ z , i ) = {u}_{n}^{′} ( z + Δ z , i ) + j \frac{βπ f Δ z}{2 {c}^{2}} ( {\sum }_{k = 1}^{n - 1} {ku}_{k}^{′} {u}_{n - k}^{′} + {\sum }_{k = n}^{N} {nu}_{k}^{′} {u}_{k - n}^{′} ) \\ n = 1 , 2 , … , N\end{matrix} \qquad ( 1 )$$

The symbol uₙ denotes a result of linear and nonlinear propagating at nf frequency (n is an integer). The symbol u′ₙ denotes a result of linear propagating at nf frequency. The fundamental frequency is denoted by f, and β is a parameter representing the nonlinearity of the propagating medium. The symbol c is the sound velocity, i represents the coordinates, and Δz is a distance variable of nonlinear propagating. The nonlinear propagating is due to the multiplicative interactions among the spectral components. In the right-hand side of Eq. (1),

$${\sum }_{k = 1}^{n - 1} {ku}_{k}^{′} {u}_{n - k}^{′ \cdot } ,$$

which is called frequency-sum component, represents the contribution of sinusoid pairs whose sum-frequency is nf.

$${\sum }_{k = n}^{N} {nu}_{k}^{′} {u}_{k - n}^{′ \cdot } ,$$

which is called frequency-difference component, represents the contribution of sinusoid pairs whose difference frequency is nf.

In the present invention, the emitting signal includes the fundamental and 3f₀ frequency components. The produced second harmonic signal mainly consists of the frequency-sum component obtained from the fundamental transmit signal and the frequency-difference component obtained from the fundamental and 3f₀ transmit signal. Because the produced second harmonic signal can be taken as the multiplicative interactions in time domain, the following Eq. (2) can be used to explain the multiplication of the fundamental signal with itself and Eq. (3) explains the interactions between the fundamental and 3f₀ signal to produce 2f₀ signal.

$$\begin{matrix}cos ( 2 π {f}_{0} t ) \times cos ( 2 π {f}_{0} t ) = \frac{1}{2} ( 1 + cos ( 2 π ( 2 {f}_{0} ) t ) ) & ( 2 ) \\ cos ( 2 π {f}_{0} t ) \times cos ( 2 π ( 3 {f}_{0} ) t ) = \frac{1}{2} [ cos ( 2 π ( 4 {f}_{0} ) t ) + cos ( 2 π ( 2 {f}_{0} ) t ) ] & ( 3 )\end{matrix}$$

In Eq. (1), it can be known that both the frequency-sum component and the frequency-difference component are sensitive to the phase of their constitutive spectral signals. For a case that u′ₖ and u′ₙ₋ₖ are both the fundamental signal, θ represents the phase of the fundamental signal, i.e. u′ₖ=uₙ₋ₖ=e^{jθ}. Therefore, the phase of the frequency-sum component is u′ₖ×u′ₙ₋ₖ=e^{j2θ}. Similarly, for another case that u′ₖ is the 3f₀ signal and u′ₖ₋ₙ is the fundamental signal, φ represents the relative phase difference between the fundamental and 3f₀ signal, i.e. u′ₖ=e^{j(θ+φ)}, u′ₖ₋ₙ=e^{jθ}. Therefore, the phase of the frequency-difference component is u′ₖ×(u′ₖ₋ₙ)*=e^{j(θ+φ)}×(e^{jθ})*=e^{jφ}. Hence, the amplitude of harmonic signals is adjustable through altering the relative phase difference between the frequency-sum and frequency-difference component. The amplitude can be not only enhanced, but also suppressed. For enhancement of the second harmonic amplitude, the two components should be in-phase to be constructively combined while the relative phase comes to zero degree (2θ=φ). In this case, the phase of the 3f₀ transmit signal should triple the phase of the fundamental transmit signal. For second harmonic suppression, the phase of the frequency-sum and the frequency-difference component should be 180 degrees out of phase (2θ=φ+π). Above all, it can be known that there is a 180 degree phase difference of the 3f₀ transmit signal between the cases of enhancement and suppression.

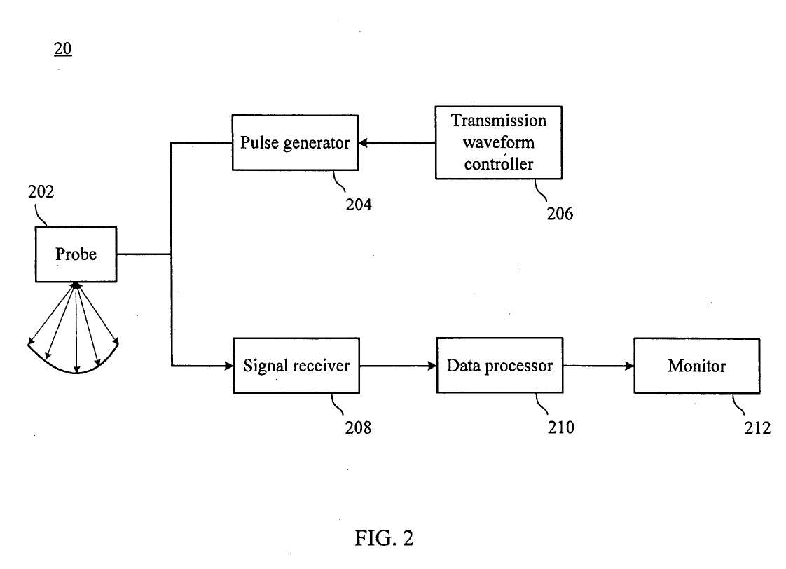

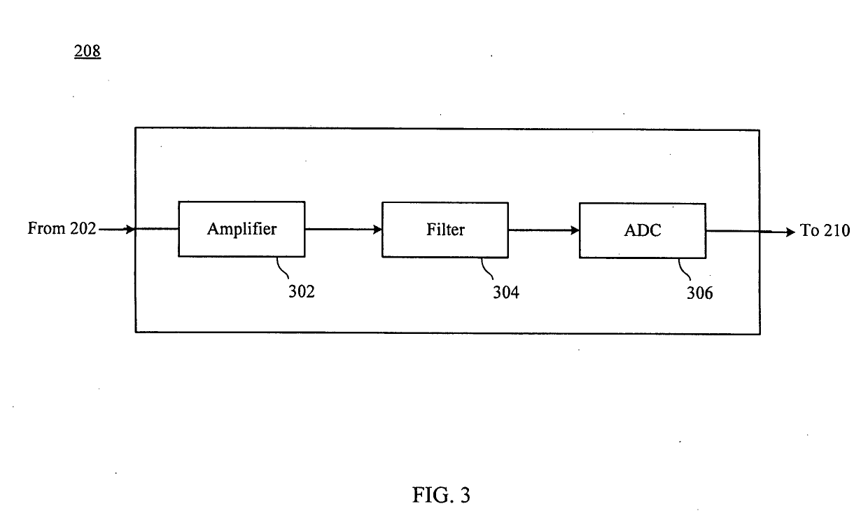

A preferred embodiment of the present invention will be provided. Referring to FIG. 2, the present invention will be illustrated with the following detailed description. An ultrasonic diagnostic apparatus 20 comprises a probe 202, a pulse generator 204, a transmission waveform controller 206, a signal receiver 208, a data processor 210 and a monitor 212. As shown in FIG. 3, the signal receiver 208 comprises an amplifier 302, a filter 304 and an analog-to-digital converter (ADC) 306.

The probe 202 may comprise a plurality of emitting elements (not shown) and receiving elements (not shown) arranged in a one-dimensional or two-dimensional manner. The emitting elements (not shown) are used to emit ultrasonic signals. The receiving elements (not shown) are used to receive echo signals and then generate electronic signals.

The pulse generator 204 supplies a drive voltage to the probe 202. According to the drive voltage supplied by the pulse generator 204, the emitting elements of the probe 202 generate corresponding amplitude of ultrasonic signals. In addition, the pulse generator 204 controls the timing of supplying the drive voltage so that the ultrasonic signals can be formed into pulse waveform. Moreover, the pulse generator 204 can control the ultrasonic pulse signals to be directed/focused in a two-dimensional direction or three-dimensional manner.

The transmission waveform controller 206 is electrically coupled with the pulse generator 204. In addition, the controller 206 controls time-sequential operations of the pulse generator 204. Moreover, the controller 206 controls the generator 204 to make the probe 202 generate the ultrasonic pulse signals with various kinds of phases, where each phase means that between fundamental and 3f₀ transmit signal. A set of an ultrasonic pulse signal with the various phases is independently relevant to a time-sequential operation. A plurality of scanning lines configures an image of one frame, where each scanning line is made up of a plurality of the time-sequential operations.

The ultrasonic pulse signals emitted by the emitting elements (not shown) of the probe 202 are nonlinearly propagated in an object (for example, a tissue in a body) so as to generate tissue harmonic signals. The fundamental transmit signals will be reflected/scattered backwardly. Therefore, the receiving elements (not shown) of the probe 202 will receive both the tissue harmonic signals and the reflected/scattered fundamental transmit signals. Finally, the received signals are converted into the electronic signals and then transmitted to the signal receiver 208.

The signal receiver 208 uses the amplifier 302 to amplify the received electronic signals, and the filter 304 filters out the fundamental transmit signals so that only the tissue harmonic signals are left. The ADC 306 converts the analog signals into digital signals. The converted signals are transmitted to the data processor 210 for processing. It may be in a sequence that the received signals are amplified with the amplifier 302, converted with the ADC 306, filtered with the filter 304, and then transmitted to the data processor 210.

The data processor 210 is electrically coupled with the signal receiver 208. The data processor 210 processes the digital signals from the signal receiver, makes up an image with a plurality of scanning lines, and makes all the images displayed on the monitor 212.

The mechanism of the above-mentioned ultrasonic diagnostic apparatus 20 in the embodiment of the present invention is described as below. The transmission waveform controller 206 controls a pulse signal to form a combinative waveform, which comprises a fundamental (f₀) frequency component and a 3f₀ frequency component. In addition, the controller 206 controls a phase difference between a fundamental transmit signal and a 3f₀ transmit signal. The phase of 3f₀ transmit signal can be adjusted relative to the phase of fundamental transmit signal by the controller 206. The phase of 3f₀ transmit signal can be set to a value that leads the amplitude of second harmonic signal to a relative maximum. For example, when the phase of 3f₀ transmit signal approaches triple the phase of fundamental transmit signal, the second harmonic signal is enhanced maximally. At this time, the phase of 3f₀ transmit signal is then adjusted to an angle, for example, about 180 degrees. The second harmonic signal is thereby suppressed maximally. In addition, the controller 206 can adjust the phase of 3f₀ transmit signal and the phase of fundamental transmit signal to maximally suppress the second harmonic signal according to the relationship, 2θ=φ+π, where θ denotes the phase of fundamental transmit signal and φ denotes the phase difference between the fundamental transmit signal and the 3f₀ transmit signal. Depending on situations, the controller 206 can adjust the phase of 3f₀ transmit signal and the phase of fundamental transmit signal to make different levels of enhancement or suppression so as to get the best image quality. The controller 206 controls the generator 204 to make the probe 202 generate the ultrasonic pulse signals with various kinds of phase difference, where each phase difference means that the phase difference between the fundamental transmit signal and the 3f₀ transmit signal. After the ultrasonic pulse signals is reflected/scattered backwardly by an object (for example, a tissue in a body), the receiving elements of the probe 202 receive the reflected/scattered fundamental transmit signals and tissue harmonic signals. At this time, the second harmonic signals are enhanced or suppressed. The received signals are converted into the electronic signals and then transmitted to the signal receiver 208. The signal receiver 208 amplifies the electronic signals, filters out the reflected/scattered fundamental transmit signals, converts into the digital signals, and transmits them to the processor 210. The data processor 210 processes the digital signals to display on the monitor 212.

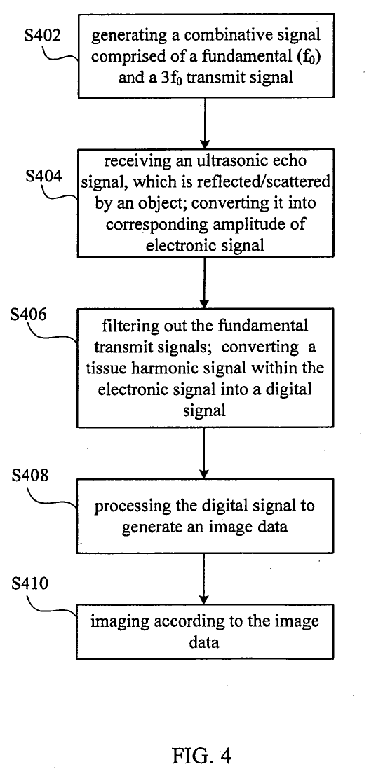

Please refer to FIG. 4. An ultrasonic imaging method according to the present invention comprises the following steps.

In step S402, it is to generate a combinative signal comprised of a fundamental transmit signal and a 3f₀ transmit signal, which the focal frequency are fundamental (f₀) and 3f₀ respectively. A plurality of combinative signals will form ultrasonic pulse signals. After emitting the ultrasonic pulse signals to an object, the object is scanned regionally.

In step S404, it is to receive ultrasonic echo signals, which are reflected/scattered by the object. The ultrasonic echo signals comprise fundamental transmit signals and tissue harmonic signals. The received echo signals are converted into corresponding amplitude of electronic signals.

In step S406, it is to filter out the fundamental transmit signals within the electronic signals, and convert the tissue harmonic signals within the electronic signals into digital signals.

In step S408, it is to process the digital signals to generate image data of the scanned object.

Finally, in step S410, it is to image according to the image data.

In addition, in step S402, the phase of 3f₀ transmit signal can be adjusted relative to the phase of fundamental transmit signal. The phase of 3f₀ transmit signal can be adjusted to a value, which leads the amplitude of second harmonic signal to a relative maximum.

Moreover, in step S402, when the phase of 3f₀ transmit signal approaches triple the phase of fundamental transmit signal, the second harmonic signal is enhanced maximally. At this time, the phase of 3f₀ transmit signal is then adjusted to an angle, for example, about 180 degrees. The second harmonic signal is thereby suppressed maximally.

Moreover, in step S402, in the beginning, the phase of 3f₀ transmit signal and the phase of fundamental transmit signal can be adjusted to maximally suppress the second harmonic signal according to a relation, 2θ=φ+π, where θ denotes the phase of fundamental transmit signal and φ denotes the phase difference between the fundamental transmit signal and the 3f₀ transmit signal.

Moreover, in step S402, the phase of 3f₀ transmit signal and the phase of fundamental transmit signal can be adjusted at any moment to make different levels of enhancement or suppression so as to get the best image quality.

Moreover, in step S406, before filtering out the reflected/scattered fundamental transmit signals, the received electronic signals can be amplified firstly.

The emitting element (not shown) and receiving element (not shown) of the probe 202 can be an emitting receiving element (not shown) having an emitting function and a receiving function. When the pulse generator 204 supplies a drive voltage to the probe 202, the emitting receiving element (not shown) generates the corresponding amplitude of ultrasonic signals. When echo signals are transmitted back to the probe 202, the emitting receiving element (not shown) can convert the echo signals into electronic signals and then transmit to the signal receiver 208. In addition, it can be employed with two probes. One of the two probes has emitting elements (not shown). The other has receiving elements (not shown). The two probes is used for emitting and receiving signals respectively.

The emitting element (not shown) and receiving element (not shown) of the probe 202 are oscillators, which can be employed with piezoelectric material. When the piezoelectric material is pressured, the lattices inside the material will collapse and electrons will flow. Electronic signals are thereby generated. Conversely, electronic voltages can make the thickness of piezoelectric material change. When the thickness of material is changed, sound wave is produced. Traditionally, the piezoelectric materials such as quartz and ceramic materials are adopted. Lead zirconate titanated (PZT) material is a common use at present.

The transmission waveform controller 206 can control to generate various kinds of wave shapes, for example, sinusoidal wave, Gaussian wave and triangular wave etc. The ultrasonic pulse signals in the aforesaid waveforms are to be emitted with the probe 202, received with the signal receiver 208 and processed with the data processor 210.

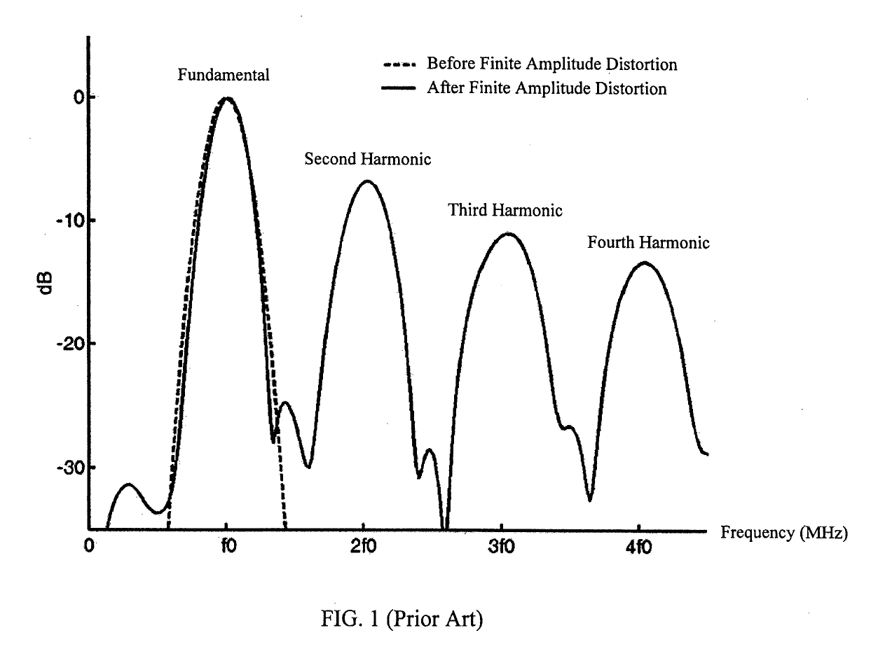

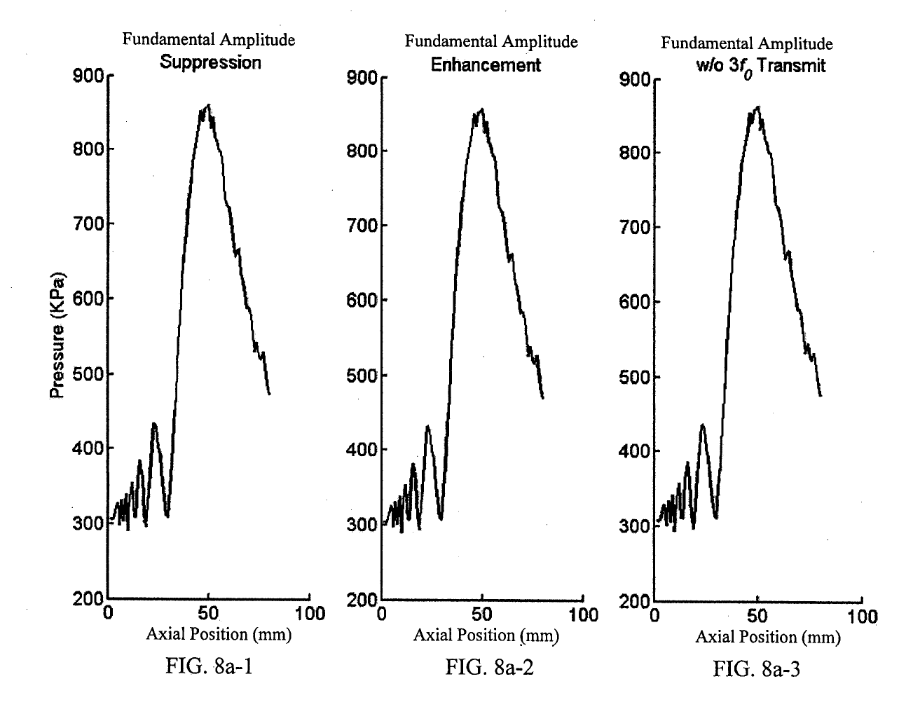

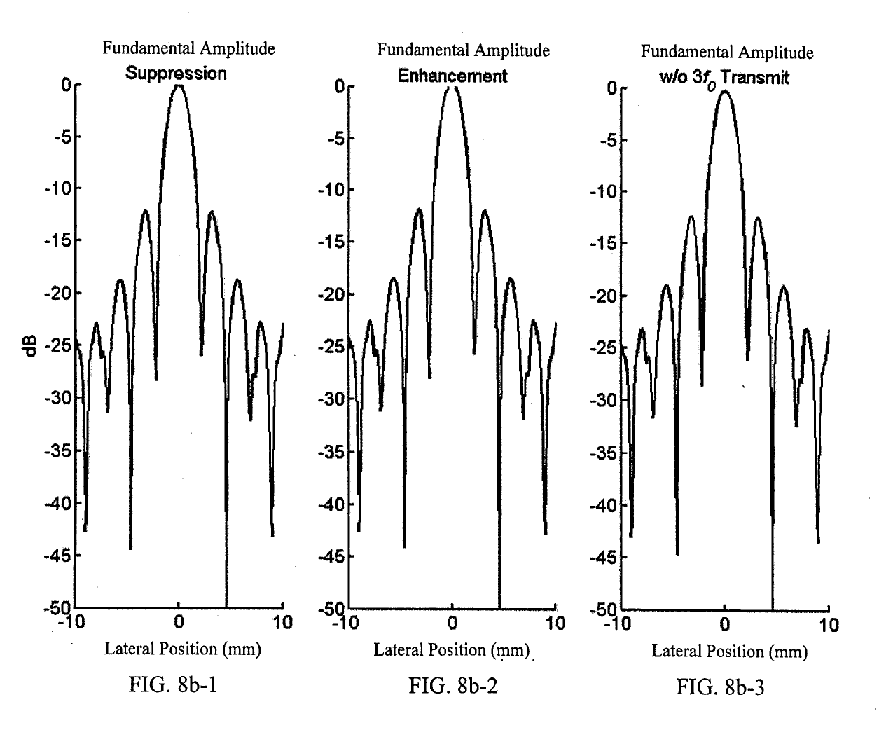

The simulations and experimental results in accordance with the present invention will be described in accompany with FIGS. 5a, 5b, 6, 7a, 7b, 8a-1, 8a-2, 8a-3, 8b-1, 8b-2, and 8b-3.

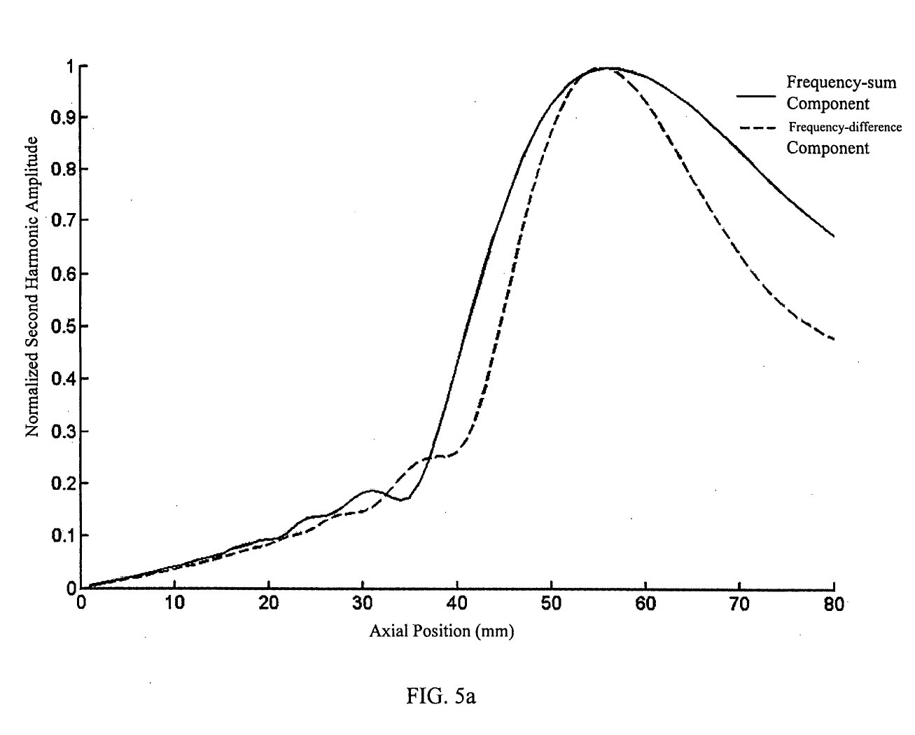

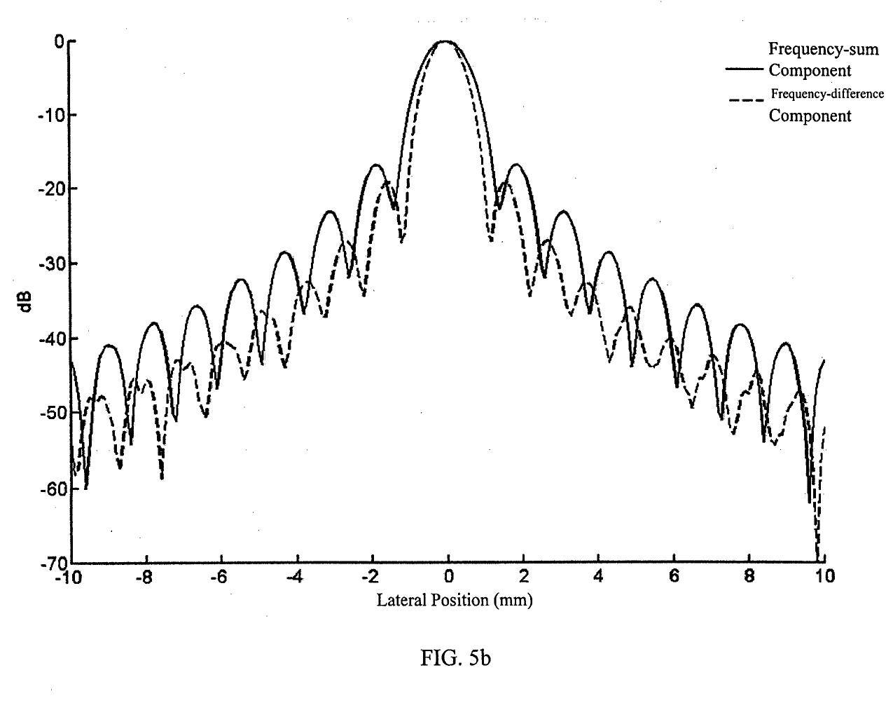

It is shown in FIG. 5a for comparing the depth of focus of the frequency-difference component and that of the frequency-sum component in the axial position. The so-called depth of focus means that sound wave remains constant amplitude within a range of depth. If the sound field is strong enough to cover a wide range, it means that the depth of focus is deeper, or better. It leads to wider range of signal-to-noise ratio (SNR) for ultrasonic images. The depth of focus of the frequency-difference component is shorter than that of the frequency-sum component because of the highly focused 3f₀ transmit signal.

Moreover, the position perpendicular to the axial position is called lateral position. It is shown in FIG. 5b for observing the mainlobe and sidelobes on the bean patterns of the second harmonic signals. Generally, signals with high frequency component lead to better focusing ability and image contrast. It represents better focusing ability when the width of mainlobe is narrower. It represents better image contrast when the amplitude of sidelobes is lower. As shown in FIG. 5b, the frequency-difference component has relatively narrower mainlobe and lower sidelobes because of the highly focused 3f₀ transmit signal.

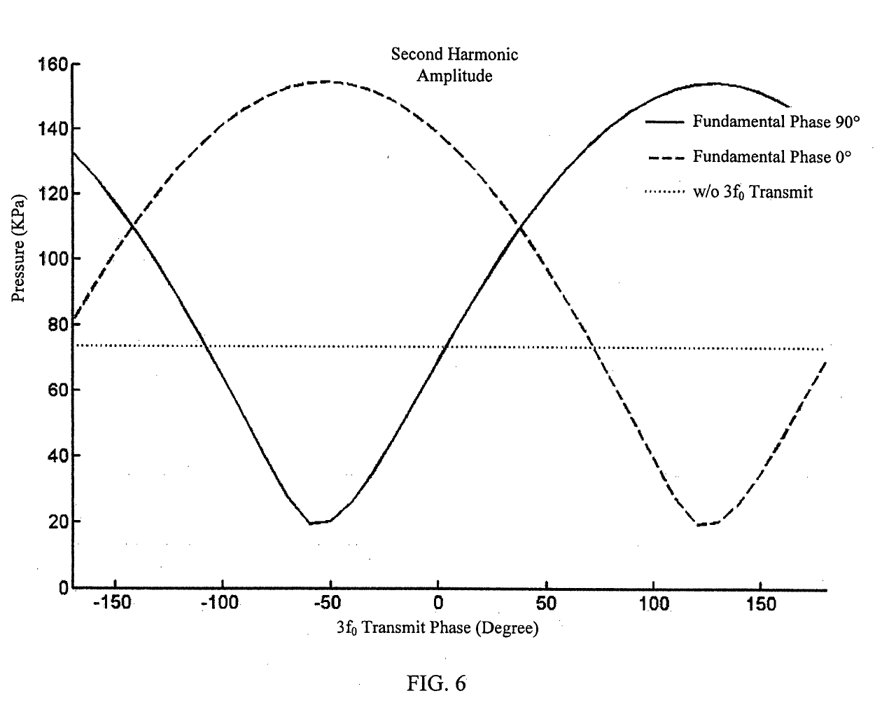

Moreover, it is shown in FIG. 6 that the variation of the second harmonic amplitude with 3f₀ transmit phase by the solid line and the dashed line. The second harmonic amplitudes produced without any transmit signal at 3f₀ frequency is depicted by the dotted horizontal line. When the fundamental transmit phase is changed from the original 90 to 0 degree, the corresponding variation of focal second harmonic amplitude also is demonstrated using the dashed line in FIG. 6. In this case, the phase for maximal enhancement is shifted from 120 to −60 degrees. The 180 degree phase shift is reasonable because the change of 3f₀ transmit phase should double the change of the fundamental transmit phase to keep the frequency-difference component in-phase with the frequency-sum component.

Moreover, the axial amplitudes of the second harmonic signal and the fundamental signal with or without optimal enhancement and optimal suppression are shown in FIG. 7a and FIGS. 8a-1, 8a-2, 8a-3, respectively. Results using the conventional transmit method is provided for comparison. It is obvious that the method of 3f₀ transmit phasing effectively changes the amplitude of the second harmonic signal for a wide range of axial depths. For both enhancement and suppression, the performance of 3f₀ transmit phasing is maximal in the focal zone in which the fundamental transmit energy and 3f₀ transmit energy can be efficiently mixed to produce the frequency-difference component. In addition, for harmonic enhancement, the depth of focus of the tissue harmonic signal also becomes shorter due to the relatively strong focusing of the frequency-difference component. It is also noticeable that the inclusion of 3f₀ transmit signal has nearly no influence on the axial fundamental amplitude in FIGS. 8a-1 and 8a-2.

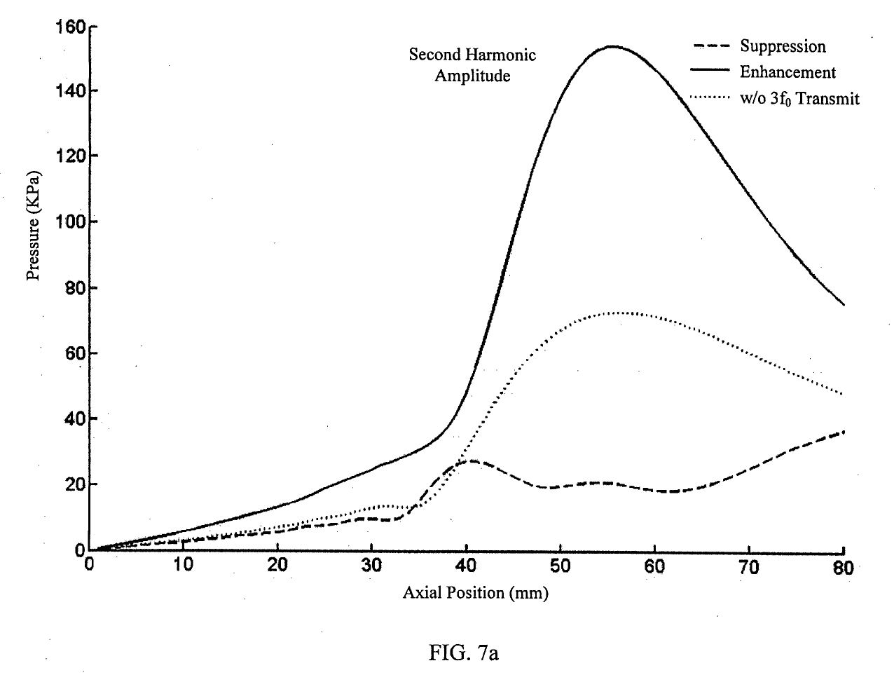

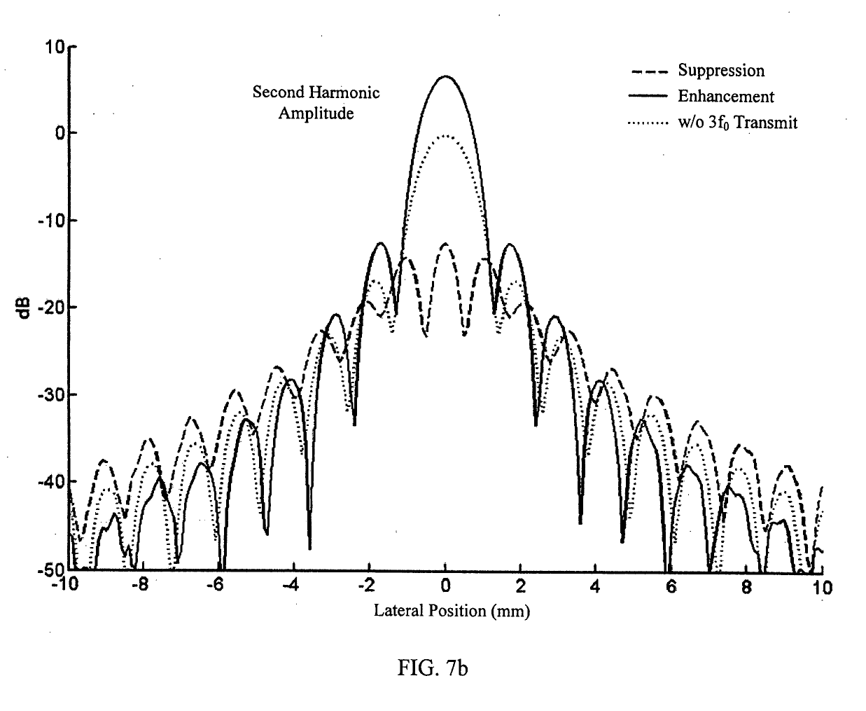

The radiation patterns of the second harmonic signal also are compared in FIG. 7b, and the fundamental radiation patterns are shown in FIGS. 8b-1, 8b-2, and 8b-3. Please note that the patterns in FIG. 7b and FIGS. 8b-1, 8b-2 are normalized to the case without 3f₀ transmit. For harmonic enhancement, the mainlobe of the radiation beam pattern remains similar to that without 3f₀ transmit, and the sidelobe levels are reduced. The reduction of sidelobe levels is a consequence of the lower sidelobes of the frequency-difference radiation pattern. In other words, with the increase in SNR of tissue harmonic signal, the spatial resolution in the lateral direction is kept unchanged, and the contrast resolution also is improved. On the contrary, for the case of harmonic suppression, sidelobe levels of the second harmonic radiation pattern are relatively elevated. Nevertheless, it should be noted that the elevated sidelobes have less effect on image quality in contrast harmonic imaging because the acoustic illumination of the microbubbles is dominated by the fundamental transmit beam.

As shown in FIGS. 8a-1, 8a-2, 8a-3 and FIGS. 8b-1, 8b-2, 8b-3, the fundamental beam remains unchanged in the presence of the 3f₀ transmit signal; hence, the generation of contrast harmonic signal is not affected. Consequently, the CTR with 3f₀ transmit phasing can be improved because of the suppression of tissue harmonic signal in the background.

While the preferred embodiment of the present invention have been illustrated and described in detail, various modifications and alterations can be made by persons skilled in this art. The embodiment of the present invention is therefore described in an illustrative but not restrictive sense. It is intended that the present invention should not be limited to the particular forms as illustrated, and that all modifications and alterations which maintain the spirit and realm of the present invention are within the scope as defined in the appended claims.

What is claimed is:

1. An ultrasonic diagnostic apparatus for emitting an ultrasonic pulse signal to scan a biological sample wherein the ultrasonic pulse signal is reflected/scattered backwardly by the biological sample to result in an ultrasonic echo signal, the ultrasonic diagnostic apparatus comprising:
a transmission waveform controller for generating a combinative signal and generating a drive voltage according to the combinative signal, wherein the combinative signal comprised of a fundamental transmit signal and a 3f₀ transmit signal, which the focal frequency are fundamental (f₀) and 3f₀ respectively;
a pulse generator for generating a pulse signal according to the drive voltage, wherein the pulse signal is converted to the ultrasonic pulse signal by piezoelectric materials;
a signal receiver for receiving the ultrasonic echo signal, which comprises the reflected/scattered fundamental transmit signal and an harmonic signal; and
a data processor for processing the harmonic signal of the ultrasonic echo signal to generate an image data

2. The ultrasonic diagnostic apparatus of claim 1 further comprising a probe electrically coupled to the pulse generator and the signal receiver, wherein the probe comprising:
an emitting element for emitting the ultrasonic pulse signal according to the pulse signal generated by the pulse generator; and
a receiving element for receiving the ultrasonic echo signal.

3. The ultrasonic diagnostic apparatus of claim 2, wherein the emitting element and the receiving element are made into the same element for emitting the ultrasonic pulse signal and receiving the ultrasonic echo signal at different moment.

4. The ultrasonic diagnostic apparatus of claim 1, wherein the phase of the 3f₀ transmit signal is adjusted relative to the phase of the fundamental transmit signal by the transmission waveform controller; when the phase of the 3f₀ transmit signal is adjusted to a first emitting phase, it leads the amplitude of a second harmonic signal of the harmonic signal to a relative maximum.

5. The ultrasonic diagnostic apparatus of claim 4, wherein the first emitting phase is substantially equal to triple the phase of the fundamental transmit signal.

6. The ultrasonic diagnostic apparatus of claim 4, wherein when the first emitting phase is further adjusted an out-phase degree into a second emitting phase, it leads the amplitude of the second harmonic signal of the harmonic signal to a relative minimum.

7. The ultrasonic diagnostic apparatus of claim 6, wherein the out-phase degree is substantially equal to 180 degrees.

8. The ultrasonic diagnostic apparatus of claim 1, wherein the phase of the 3f₀ transmit signal is adjusted relative to the phase of the fundamental transmit signal by the transmission waveform controller so that a relation between the phase of the 3f₀ transmit signal and the phase of the fundamental transmit signal substantially is 2θ=φ+π, where θ denotes the phase of the fundamental transmit signal and φ denotes the phase difference between the fundamental transmit signal and the 3f₀ transmit signal.

9. The ultrasonic diagnostic apparatus of claim 1, wherein the transmission waveform controller is further used for generating different kinds of wave shapes.

10. The ultrasonic diagnostic apparatus of claim 9, wherein the wave shapes generated by the transmission waveform controller are selected from a group consisting of a sinusoidal wave, an Gaussian wave and a triangular wave.

11. The ultrasonic diagnostic apparatus of claim 1, wherein the signal receiver comprises an amplifier for amplifying the ultrasonic echo signal.

12. The ultrasonic diagnostic apparatus of claim 1, wherein the signal receiver comprises a filter for filtering out the fundamental transmit signal of the ultrasonic echo signal, and remaining the harmonic signal of the ultrasonic echo signal.

13. An ultrasonic imaging method utilized to an ultrasonic pulse signal to scan a biological sample wherein the ultrasonic pulse signal is reflected/scattered backwardly by the biological sample to result in an ultrasonic echo signal, the ultrasonic imaging method comprising:
generating a combinative signal, wherein the combinative signal comprised of a fundamental transmit signal and a 3f₀ transmit signal, which the focal frequency are fundamental (f₀) and 3f₀ respectively;
converting the combinative signal to the ultrasonic pulse signal, wherein after emitting the ultrasonic pulse signal, the biological sample is scanned regionally;
receiving the ultrasonic echo signal, which comprises the reflected/scattered fundamental transmit signal and an harmonic signal; and
processing the harmonic signal of the ultrasonic echo signal to generate an image data.

14. The ultrasonic imaging method of claim 13 further comprising: adjusting the phase of the 3f₀ transmit signal relative to the phase of the fundamental transmit signal; when the phase of the 3f₀ transmit signal is adjusted to a first emitting phase, it leads the amplitude of a second harmonic signal of the harmonic signal to a relative maximum.

15. The ultrasonic imaging method of claim 14, wherein the first emitting phase is substantially equal to triple the phase of the fundamental transmit signal.

16. The ultrasonic imaging method of claim 14, wherein when the first emitting phase is further adjusted an out-phase degree into a second emitting phase, it leads the amplitude of the second harmonic signal of the harmonic signal to a relative minimum.

17. The ultrasonic imaging method of claim 16, wherein the out-phase degree is substantially equal to 180 degrees.

18. The ultrasonic imaging method of claim 13 further comprising: adjusting the phase of the 3f₀ transmit signal relative to the phase of the fundamental transmit signal so that a relation between the phase of the 3f₀ transmit signal and the phase of the fundamental transmit signal substantially is 2θ=φ+π, where θ denotes the phase of the fundamental transmit signal and φ denotes the phase difference between the fundamental transmit signal and the 3f₀ transmit signal.

19. The ultrasonic imaging method of claim 13 further comprising: amplifying the ultrasonic echo signal.

20. The ultrasonic imaging method of claim 13 further comprising: filtering out the fundamental transmit signal of the ultrasonic echo signal, and remaining the harmonic signal of the ultrasonic echo signal.
